(19) United States
(12) Patent Application Publication
(10) Pub. No.: US 2023/0293075 A1
(43) Pub. Date: Sep. 21, 2023

(54) BIOMAGNETIC MEASURING APPARATUS, BIOMAGNETIC MEASURING SYSTEM, AND BIOMAGNETIC MEASURING METHOD

(71) Applicants: Haruka KURIHARA, Ishikawa (JP); Yoshinori OKADA, Kanagawa (JP); Hiroshi KUBOTA, Ishikawa (JP); Makoto INAMOTO, Ishikawa (JP); Nobuhiro SHIMA, Kanagawa (JP)

(72) Inventors: Haruka KURIHARA, Ishikawa (JP); Yoshinori OKADA, Kanagawa (JP); Hiroshi KUBOTA, Ishikawa (JP); Makoto INAMOTO, Ishikawa (JP); Nobuhiro SHIMA, Kanagawa (JP)

(73) Assignee: Ricoh Company, Ltd., Tokyo (JP)

(21) Appl. No.: 18/175,876

(22) Filed: Feb. 28, 2023

(30) Foreign Application Priority Data
Mar. 15, 2022 (JP) 2022-040488

(51) Int. Cl. A61B 5/248; A61B 6/04; A61B 90/00; A61B 6/00

(52) U.S. Cl. CPC A61B 5/248; A61B 6/0407; A61B 90/39; A61B 6/5247; A61B 2090/3966

(57) ABSTRACT

A biomagnetic measuring apparatus includes a biomagnetic detector, a first marker configured to be detectable by the biomagnetic detector, a radiation source, a radiation detector provided to face the radiation source, and a second marker configured such that an image of the second marker can be captured by the radiation source and the radiation detector. Positional information about the first marker and the second marker is known or obtainable.

### CROSS-REFERENCE TO RELATED APPLICATIONS

This application claims priority to Japanese Patent Application No. 2022-040488 filed on Mar. 15, 2022, the entire contents of which are incorporated herein by reference.

### BACKGROUND OF THE INVENTION

### 1. Field of the Invention

The present disclosure relates to biomagnetic measuring apparatuses, biomagnetic measuring systems, and biomagnetic measuring methods.

### 2. Description of the Related Art

A biomagnetic measurement result obtained by magnetospinography does not include information related to the positional relationship of bones and nerves. Hence, a morphological image needs to be superimposed onto a magnetic field distribution based on the biomagnetic measurement result or onto a reconfigured current distribution. To superimpose the morphological image onto the biomagnetic measurement result, points (for example, signs or marks) known in the coordinate system of a biomagnetic detector need to be apparent in the morphological image. Marker coils are sometimes used as such known positional points in the coordinate system of the biomagnetic detector.

A biomagnetic measuring apparatus that performs radiographic imaging and biomagnetic measurement, without moving a test subject from a bed, by using electromagnetic coils as markers to accurately superimpose the image diagnosis result onto the biomagnetic measurement result is known (for example, see Patent Document 1). Further, a biomagnetic measuring apparatus that uses an ultrasound image to specify the positional relationship between the magnetic sensor and the position of a nerve during magnetic-field measurement is known (for example, see Patent Document 2).

In a case where marker coils, which are provided in a detection region of a magnetic sensor, are to be used as markers for the alignment of a morphological image, it may be difficult to discriminate the positions of the marker coils due to the images of the marker coils overlapping with the images of, for example, bones and joints in a measurement site. Providing the marker coils in a position that does not interfere with the measurement site, that is, in a position near the boundary of a magnetic detection region prevents magnetic fields that are generated from the marker coils from being detected sufficiently, thus reducing the accuracy of the estimation of the marker coil positions. As a result, the alignment accuracy of the biomagnetic measurement result and the morphological image decreases.

### RELATED-ART DOCUMENTS

### Patent Document

[Patent Document 1] Japanese Unexamined Patent Application Publication No. 2019-098156
[Patent Document 2] Japanese Unexamined Patent Application Publication No. 2021-151429

### SUMMARY OF THE INVENTION

According to one embodiment, a biomagnetic measuring apparatus includes a biomagnetic detector, a first marker configured to be detectable by the biomagnetic detector, a radiation source, a radiation detector provided to face the radiation source, and a second marker configured such that an image of the second marker can be captured by the radiation source and the radiation detector. Positional information about the first marker and the second marker is known or obtainable.

### BRIEF DESCRIPTION OF THE DRAWINGS

FIG. 1A is a side view of a biomagnetic measuring apparatus during magnetic measurement according to an embodiment;

FIG. 1B is a front view of the biomagnetic measuring apparatus during magnetic measurement according to the embodiment;

FIG. 2A is a side view of the biomagnetic measuring apparatus during radiographic imaging according to the embodiment;

FIG. 2B is a front view of the biomagnetic measuring apparatus during radiographic imaging according to the embodiment;

FIG. 3 is a view illustrating an example of a first marker and second markers provided on a bridge;

FIG. 4A is a view illustrating another example of the first marker and the second markers provided on the bridge;

FIG. 4B is a view illustrating an example of the positional relationship between a first region and a second region in the arrangement of FIG. 4A;

FIG. 4C is a view illustrating another example of the positional relationship between the first region and the second region in the arrangement of FIG. 4A;

FIG. 5 is a view illustrating another example of the first marker and the second marker provided on the bridge;

FIG. 6 is a view illustrating yet another example of the first marker and the second marker provided on the bridge;

FIG. 7 is a view illustrating a modification of the bridge;

FIG. 8A is a schematic view of a biomagnetic measuring system during biomagnetic measurement according to the embodiment;

FIG. 8B is a schematic view of the biomagnetic measuring system during radiographic imaging according to the embodiment;

FIG. 9 is a functional block diagram of an information processing apparatus;

FIG. 10 is a flowchart of a biomagnetic measuring method according to the embodiment; and

FIG. 11 is a more detailed flowchart of the biomagnetic measuring method according to the embodiment.

### DESCRIPTION OF THE EMBODIMENTS

An object of the present disclosure is to improve the alignment accuracy in the superimposition of a biomagnetic measurement result in biomagnetic measurement.

The alignment accuracy is improved in the superimposition of a biomagnetic measurement result in biomagnetic measurement.

Two types of markers are used in the embodiment to improve the alignment accuracy when a biomagnetic measurement result result obtained by a biomagnetic detector and an image captured by radiographic imaging are superimposed onto one another. That is, a first marker that is detectable by the biomagnetic detector and a second marker whose image can be captured by radiographic imaging are used. The first marker is provided in a first region where biomagnetic detection can be performed, and the second marker is provided in a second region where radiographic imaging can be performed. The first region and the second region have portions that overlap with each other. Positional information about the first marker and the second marker is known or obtainable. A state in which positional information is “known or obtainable” refers to a state in which the positional information can be determined using any suitable method. The positional information includes the coordinate positions of the first marker and the second marker and the relative positional relationship between them in a spatial coordinate system. Information about the coordinates and the positional relationship of the first marker and the second marker that have been measured in advanced may be stored in, for example, a given storage unit or a storage table. Alternatively, such information about the coordinates and the positional relationship of the first marker and the second marker that have been measured in advance may be obtained from the cloud or an external source via a network. The coordinates and the positional relationship of the first marker and the second marker may be ideal design values or measured values. In a case where the difference between the designed value and the measured value exceeds an allowable error, the difference may be used as correction information.

By setting a state where the positional information about the first marker that is detected by the biomagnetic detector and the second marker that is captured in a radiographic image can be known, the alignment accuracy of the biomagnetic detection result and the radiographic image can be improved even in cases where the position of the first marker is difficult to discriminate in the radiographic image. The biomagnetic measurement according to the embodiment will be described in detail hereinafter. In the following description, a repetitive description may be omitted by using the same reference symbols to denote the same components. Further, an image of a body part captured by radiographic imaging will be referred to as a “morphological image”. Furthermore, although examples in which a morphological image is superimposed onto a biomagnetic measurement result will be described hereinafter, the embodiment is also applicable to a case in which a biomagnetic measurement result is superimposed onto a morphological image.

<Basic Configuration of Biomagnetic Measuring Apparatus>

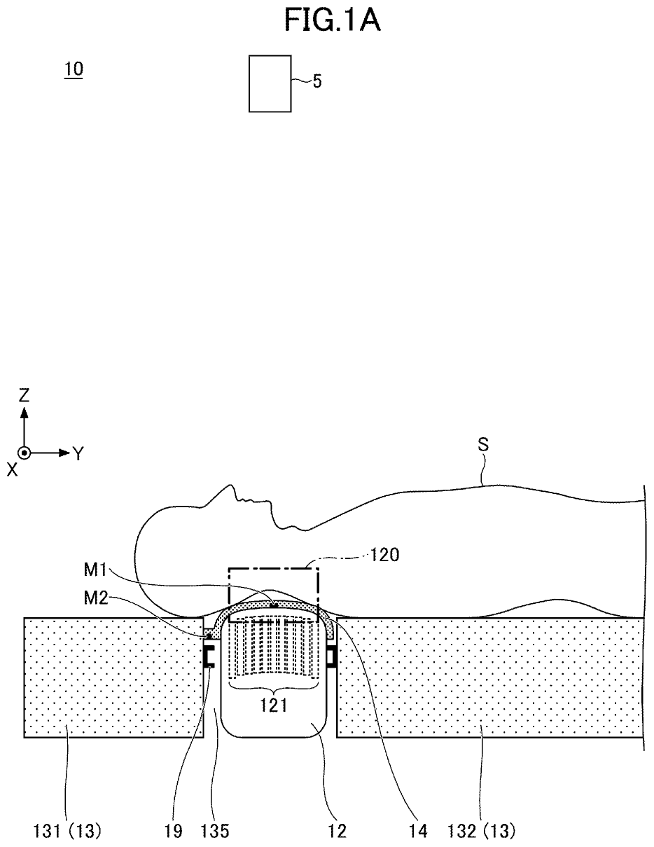

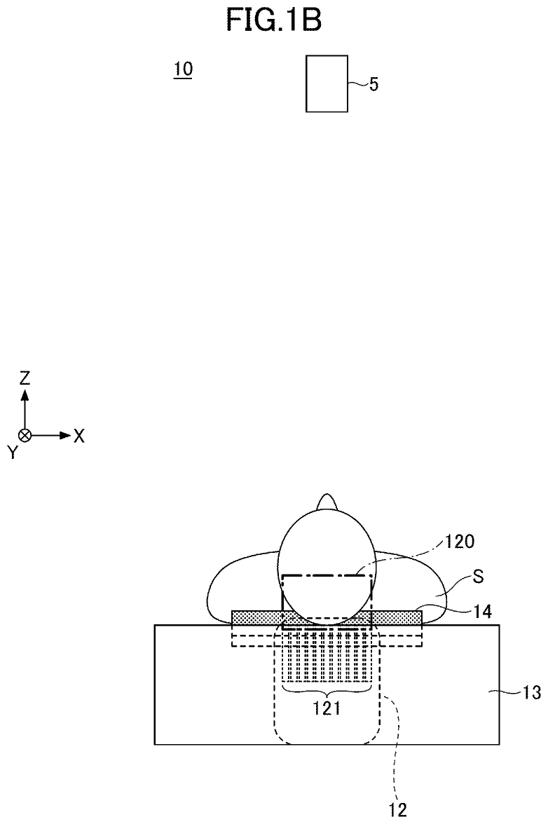

FIG. 1A is a side view of a biomagnetic measuring apparatus 10 during magnetic measurement according to the embodiment. FIG. 1B is a front view of the biomagnetic measuring apparatus 10 during magnetic measurement. In the spatial coordinate system of FIGS. 1A and 1B, a plane in which a measurement target subject S (to be simply referred to as the subject S hereinafter) lies is the X-Y plane, a height direction perpendicular to the X-Y plane is the Z direction, the width direction of a bed 13 on which the subject S lies is the X direction, and the length direction of the bed 13 is the Y direction.

The biomagnetic measuring apparatus 10 includes a biomagnetic detector 12, a first marker M1 configured to be detectable by the biomagnetic detector 12, and a second marker M2 configured such that its image can be captured by emitting radiation from a radiation source 5. The first marker M1 is provided in a first region 120 that is detectable by the biomagnetic detector 12, the second marker M2 is provided outside of the first region 120. The bed 13 configured to support the subject S is placed in a biomagnetic measurement space illustrated in FIGS. 1A and 1B. The bed 13 may include a first portion 131 that is configured to support the head side of the subject with respect to a measurement site (for example, the neck) of the subject S and a second portion 132 that is configured to support the tail side of the subject S with respect to the measurement site of the subject S. A space 135 that contains the biomagnetic detector 12 is provided between the first portion 131 and the second portion 132. A bridge 14 that is placed across the space 135 is provided on at least either the first portion 131 or the second portion 132 of the bed 13.

The position of the bridge 14 and the number of bridges 14 are not limited to those exemplified in FIG. 1A. Instead of the configuration in FIG. 1A or in addition to the configuration in FIG. 1A, a bridge may be provided in a position of the bed 13 that corresponds to the lumbar region of the subject S. In such a case, the biomagnetic detector 12 that is contained below the bridge may detect the biomagnetism caused by, for example, cauda equina nerve activity from the skin surface of the lumbar region of the subject S.

The radiation source 5 configured to emit radiation is installed above the bridge 14, that is, above the biomagnetic detector 12. The radiation source 5 emits radiation that is transmitted through a living body. X-rays, α-rays, β-rays, γ-rays, or particle beams with energies equal to these can be used as the radiation. The bridge 14 is transparent to the radiation emitted from the radiation source 5, and is made of a non-magnetic material that does not interfere with biomagnetic measurement. Forming the bridge 14 with a material that has high radiation transmittance can reduce the amount of radiation exposure to the subject S. The radiation source 5 may be installed so that the irradiation position, irradiation angle, and the like can be changed in accordance with the irradiation position or the position of the bridge 14.

To allow the bridge 14 to be situated near the biomagnetic detector 12, the bridge 14 is made of a non-magnetic material. It is desirable for the bridge 14 to be a non-metallic element in the interest of suppressing the influence of Johnson noise (thermal noise). Further, to support the measurement site of the subject S, it is desirable for the bridge 14 to have some degree of mechanical strength and an external shape that corresponds to the shape of the measurement site. The bridge 14 is made of, for example, glass fiber reinforced plastic (GFRP), polycarbonate, or ceramic and is processed into a desired shape by, for example, injection molding or cutting.

The first marker M1 and the second marker M2 are embedded in the bridge 14. The first marker M1 is provided at substantially the center of the bridge 14. The first marker M1 generates a magnetic field in response to application of a current. The first marker M1 is made of, for example, a non-magnetic metal material that has been patterned into a coil-shape. Applying a current to the coil allows the first marker M1 to function as a magnetic marker that is detectable by the biomagnetic detector 12. The coordinate position of the first marker M1 in the spatial coordinate system is known or obtainable.

The first marker M1 may be provided in the center portion of the bridge 14 or may be provided in a position other than the center of the bridge 14 as long as the magnetic field can be detected well by biomagnetic detector 12. A coiled wire may be used as the first marker M1. In a case where the first marker M1 is to be provided on the curved surface of the bridge 14, a coiled pattern formed on a flexible printed circuit (FPC) may be used as the first marker M1.

The second marker M2 is provided in a location, such as the end portion of the bridge 14, that does not interfere with the biomagnetic measurement and allows radiographic imaging using the radiation source 5 to be performed. The radiation transmittance of the second marker M2 with respect to the radiation from the radiation source 5 is lower than the radiation transmittance of the first marker M1. Hence, the second marker M2 functions as a radiation marker. The second marker M2 may be formed by, for example, a non-magnetic metal ball or a metallic pattern. The second marker M2 may be provided outside of the first region 120, which is the detection region of the biomagnetic detector 12. The coordinate position of the second marker M2 or the relative position of the second marker M2 with respect to the first marker M1 in the spatial coordinate system is known or obtainable. The first marker M1 and the second marker M2 are provided on the lower surface of the bridge 14, that is, near the surface that is opposite the biomagnetic detector 12 so as to avoid contact with the subject S.

A magnetic sensor array 121 of the biomagnetic detector 12 is provided at a position facing the bridge 14. The magnetic sensor array 121 detects the biomagnetic field emitted from the measurement site of the subject S placed on the bridge 14. In a case where the measurement site is the neck, the mutually opposing surfaces of the biomagnetic detector 12 and the bridge 14 are formed in a shape conforming to the rear side of the human cervical spine. The magnetic sensor array 121 detects the magnetic field that is generated with the neural activity of the cervical spinal cord. It is desirable for the bridge 14 to be in a shape that conforms to the surface shape of the biomagnetic detector 12 so as to allow the biomagnetic detector 12 and the bridge 14 to be in contact with each other without a gap therebetween. Although the biomagnetic signals emitted from the measurement can be detected with lower attenuation as the distance between the biomagnetic detector 12 and the measurement site of the subject S decreases, the thickness of the bridge 14 is set appropriately in balance with the mechanical strength.

The magnetic sensor array 121 is fixed to, for example, an insulated container that has a temperature adjustment function. For example, superconducting quantum interference devices (SQUID), magnetoresistive (MR) sensors, magneto-impedance (MI) sensors, optically pumped atomic magnetometers (OPAM) known as room-temperature magnetic sensors, and the like can be used as the magnetic sensors constituting the magnetic sensor array 121.

In a case where SQUID sensors are used, the magnetic sensor array 121 is housed in an insulating container called a cryostat and is cooled to a very low temperature. In a case where magnetic sensors that do not require cooling by liquid helium are used, a sensor container other than the insulated container may be used. The position of the biomagnetic detector 12 may be fixed within the measurement space to minimize the influence of magnetic field fluctuations.

By separately providing the first marker M1 and the second marker M2 whose positional information in the spatial coordinate system is known or obtainable, a morphological image that has been captured by radiographic imaging can be accurately superimposed onto a biomagnetic measurement result. The superimposition of the morphological image onto the biomagnetic measurement result will be described later.

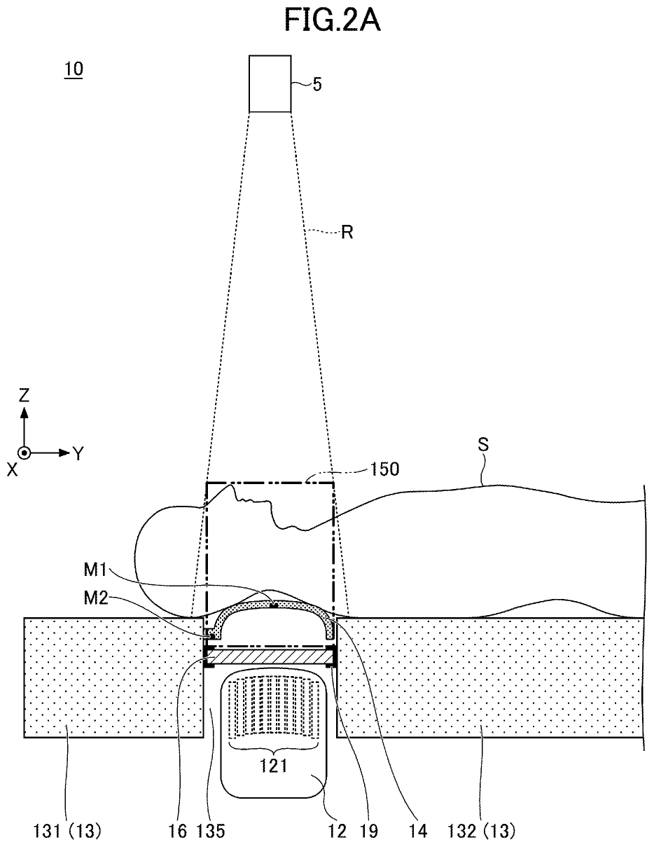

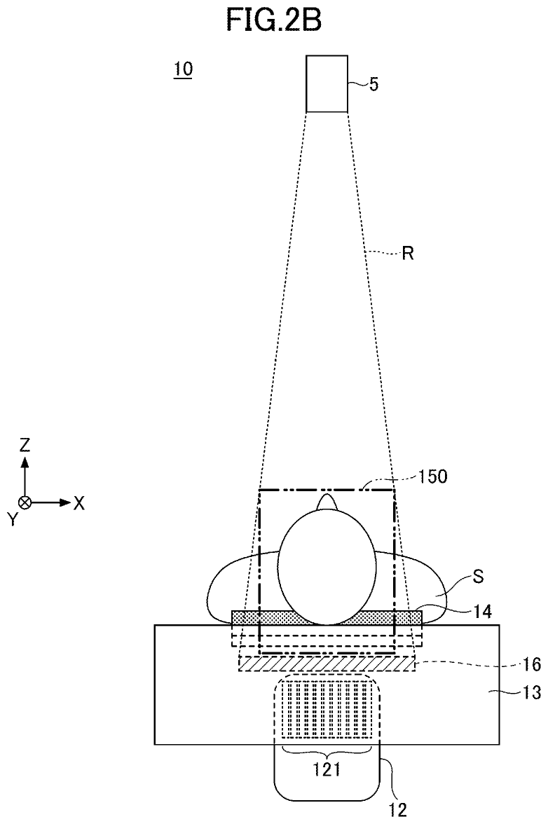

FIG. 2A is a side view of the biomagnetic measuring apparatus 10 during radiographic imaging according to the embodiment. FIG. 2B is a front view of the biomagnetic measuring apparatus 10 during radiographic imaging. The spatial coordinate system in FIGS. 2A and 2B are the same as the spatial coordinate system in FIGS. 1A and 1B. The biomagnetic measuring apparatus 10 includes the radiation detector 16 that can sense radiation emitted from the radiation source 5. Either the biomagnetic measurement by the biomagnetic detector 12 or the radiographic imaging may be performed first.

The bed 13 includes a lifting mechanism. The bed 13 is raised in the +Z direction when radiographic imaging is to be performed. The rising of the bed 13 causes the bridge 14 that is fixed to the bed 13 to also rise, thereby creating a gap between the biomagnetic detector 12 and the bridge 14. The radiation detector 16 is installed in this gap. For example, a mount 19 for the attachment of the radiation detector 16 is provided between the first portion 131 and the second portion 132 of the bed 13, and the radiation detector 16 is set to the mount 19.

In the case of X-ray imaging, the radiation detector 16 may be a flat panel detector (FPD). The FPD detects, via individual elements, the X-rays transmitted through the subject S and outputs an electrical signal corresponding to the X-ray dose. Alternatively, an imaging plate that uses a film coated with photostimulable phosphor may be used instead of the FPD. In the case of the latter, the energy of the radiation transmitted through the subject S is accumulated in the photostimulable phosphor. Irradiation by light of a specific wavelength or an electromagnetic wave is performed after imaging, and electrical signals corresponding to the amount of flash produced by the stimulus of the irradiation are obtained.

After the radiation detector 16 is set on the mount 19, an image of the measurement site is captured in the second region 150, which is the imaging region determined by the radiation source 5 and the radiation detector 16. The radiographic image of the measurement site includes the images of the first marker M1 and the second marker M2 provided in the bridge 14. In a case where the first marker M1 is provided at the center of the bridge 14, the first marker M1 may not be clearly identifiable due to the image of the first marker M1 overlapping with a bone or a joint at the measurement site. In contrast, the image of the second marker M2 provided at the end portion of the bridge 14 can be clearly captured without interfering with the radiographic image of the measurement site. The positions of the first marker M1 and the second marker M2 in the coordinate system of the biomagnetic detector 12 are already known. Hence, even in a case where the radiographic image of the first marker M1 is unclear, the morphological image can be accurately superimposed onto the biomagnetic measurement result, which is obtained by the radiographic imaging, based on the relative position of the second marker M2 with respect to the first marker M1.

In a case where biomagnetic measurement is to be performed after radiographic imaging, the radiation detector 16 is removed from the mount 19 and the bed 13 is lowered after the end of radiographic imaging. The lowering of the bed 13 causes the bridge 14 to also be lowered, thus bringing the bridge 14 to be in close contact with the upper surface of the biomagnetic detector 12. Subsequently, the biomagnetic measurement described above with reference to FIGS. 1A and 1B is performed.

The lifting mechanism of the bed 13 may be manual or electric. An electric lifting mechanism including a hydraulic cylinder and an electric pump may be used. The lifting mechanism may be provided independently for each of the first portion 131 and the second portion 132 of the bed 13 so as to move both the first portion 131 and the second portion 132 simultaneously in the Z direction or to move only one of the first portion 131 and the second portion 132 in the Z direction.

Changing the height of the bed 13 changes the scale of the radiographic image relative to the measurement result of the biomagnetic detector 12. In a case where the biomagnetic data of the measurement site are obtained at a lowered position of the bed 13 and the morphological image is obtained by radiographic imaging at a raised position of the bed 13, the amount by which the bed 13 is raised, that is, the amount of change in the height position of the bed 13 needs to be known to superimpose the morphological image onto the biomagnetic measurement result.

Hence, the magnetic field of the first marker M1 may be measured at the raised position of the bed 13 without the radiation detector 16. Although the biomagnetic detector 12 will hardly detect the biomagnetic field of the skin surface of the subject S when the bed 13 is in the raised position, the biomagnetic detector 12 can detect the magnetic field generated from the first marker M1 when the first marker M1 is energized. By measuring the magnetism of the first marker M1 both in a state where the bed is in the lowered position and in a state where the bed is in the raised position, a difference vector of the two measurement results can be calculated to determine the amount by which the bed is raised (that is, the distance the bed has been raised).

As described above, in order to superimpose the morphological image, which is obtained by radiographic imaging, onto the biomagnetic measurement result, an object whose position in the spatial coordinate system is known needs to be captured in the radiographic image. The second marker M2 can be used as an object whose position in the spatial coordinate system is known or obtainable. The second marker M2 is provided in an imaging range, that is, is, in the second region 150 that is determined based on the positional relationship between the radiation source 5 and the radiation detector 16.

The second marker M2 is made of a non-magnetic metal capable of being radiographically imaged (that is, a non-magnetic metal that has a low radiation transmittance) and does not hinder biomagnetic measurement. For example, brass, copper, or tungsten can be used for the second marker M2. In a case where automatic circle detection of X-ray images can be used, the second marker M2 may be a sphere. The sphere cannot be embedded in the bridge 14 if the sphere is too large. However, detecting the position of the sphere in the radiographic image is difficult if the sphere is too small. Hence, the sphere needs to be of a suitable size. The diameter of the sphere in a case where a sphere is used as the second marker M2 is preferably 1 mm or more and 10 mm or less or is more preferably 2 mm or more and 6 mm or less.

The first marker M1 and the second marker M2 are measured in three dimensions from the same reference point in the actual spatial coordinate system, and the relative distance between the first marker M1 and the second marker M2 is known or is obtainable. This measurement result and the result of the position estimated based on the first marker M1 can be combined to determine the positional coordinates of the second marker M2 in the spatial coordinate system.

<Arrangement of Markers>

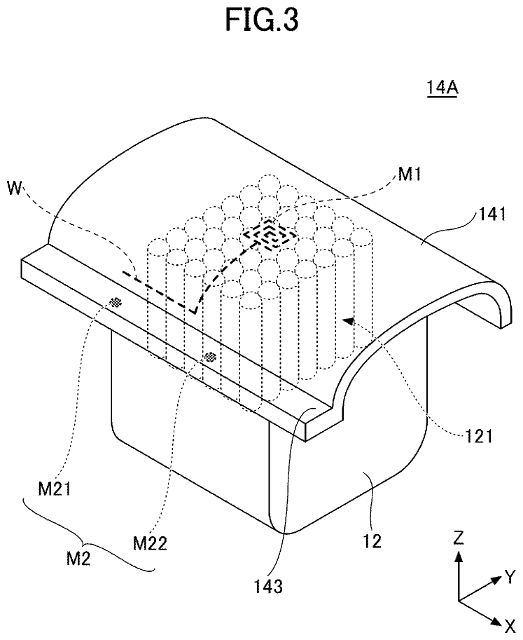

FIG. 3 illustrates an example of the first marker M1 and the second marker M2 that are provided in a bridge 14A. The bridge 14A includes a body 141 that faces the biomagnetic detector 12, and a flat portion 143 that extends from the body 141. The body 141 may include a curved surface that conforms to the shape of the upper surface of the biomagnetic detector 12. The flat portion 143 may serve as a portion to be fixed to the bed 13.

The first marker M1 is provided at substantially the center of the body 141. Wiring W for supplying current is connected to the first marker M1. A portion of the wiring W is embedded together with the first marker M1 in the bridge 14A and is connected to an external current source. Although the wiring W is depicted using a single dotted line for the sake of descriptive convenience in FIG. 3, the wiring W may include two wires connected to both ends of the coil forming the first marker M1.

The second marker M2 is embedded in the flat portion 143 of the bridge 14A. The second marker M2 includes two markers M21 and M22 (a plurality of second markers) that are provided at a predetermined distance away from each other. Both the markers M21 and M22 may be non-magnetic metal spheres. In such a case, the markers M21 and M22 appear as two circles or dots in the radiographic image. Let D1 (for example, 50 mm) be a center-to-center distance between the marker M21 and the marker M22 on the flat portion 143 and D2 (for example, 80 mm) be a center-to-center distance between two circles in the radiographic image. When a morphological image obtained by radiographic imaging is superimposed onto a magnetic field distribution, the morphological image is enlarged or reduced to D1/D2 in accordance with the biomagnetic measurement result.

The positional relationship of the marker M21 and the marker M22 with respect to the first marker M1 is known. After the radiographic image has been enlarged or reduced, the markers M21 and M22 in the morphological image can be relatively aligned with respect to the position of the first marker M1 that is detected in the biomagnetic measurement. As a result, the radiographic image can be accurately superimposed onto the biomagnetic measurement result.

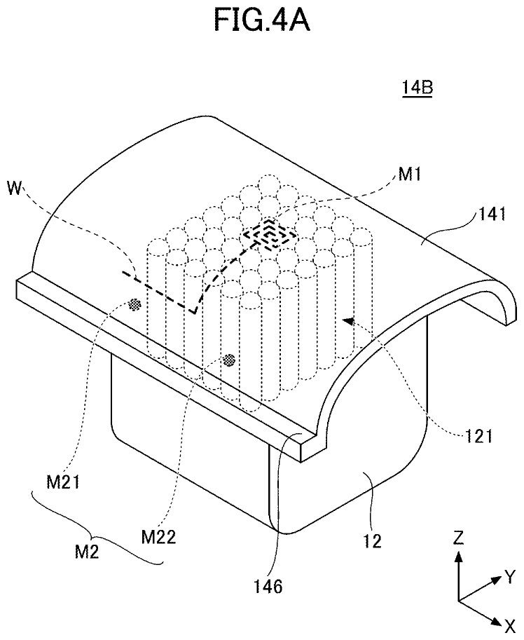

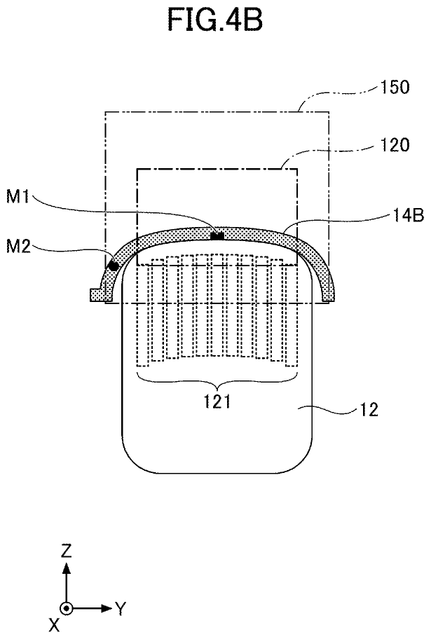

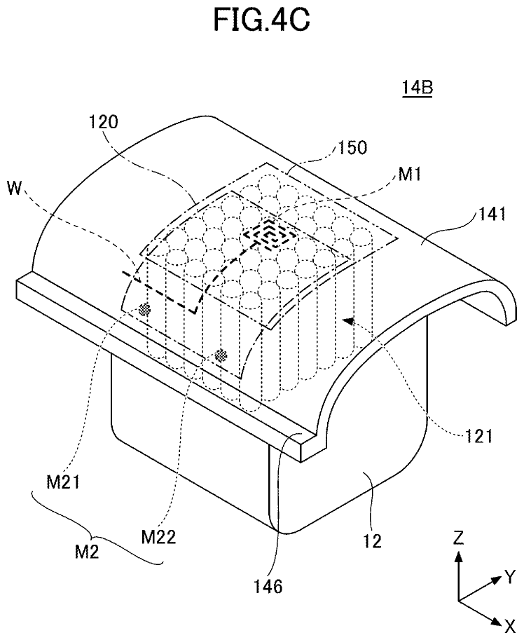

FIG. 4A illustrates another example of the first marker M1 and the second marker M2 provided in a bridge 14B. The bridge 14B includes the body 141 that faces the biomagnetic detector 12 and a fixing portion 146 that extends from the body 141. The body 141 may include the curved surface that conforms to the shape of the upper surface of the biomagnetic detector 12. The fixing portion 146 has a shape and a dimension that enable it to be fixed to the bed 13.

In FIG. 4A, both the first marker M1 and the second marker M2 are provided in the body 141. For example, the first marker M1 is provided at substantially the center of the body 141 where magnetic detection sensitivity is favorable. The second marker M2 is embedded in an end portion of the body 141 so as to be positioned outside of the magnetic detection region. In a similar manner to FIG. 3, the second marker M2 may include the two markers M21 and M22 provided a predetermine distance away from each other.

FIG. 4B illustrates an example of the positional relationship between the first region 120 and the second region 150 in the arrangement illustrated in FIG. 4A. The first region 120 is the detection range of the magnetic sensor array 121 of the biomagnetic detector 12. The second region 150 is the imaging range determined based on the positional relationship between the radiation source 5 and the radiation detector 16. The second marker M2 is positioned so as to be outside of the first region 120, but within the second region 150. In a plane (that may be flat or curved as long it is a two-dimensional plane) parallel to the magnetic detection surface or the radiographic imaging surface, the area of the first region 120 is smaller than the area of the second region 150. The first marker M1 is provided in the first region 120, and the second marker M2 is provided so as to be outside of the first region 120 and inside of the second region 150. As a result, an image of the second marker M2 can be captured without the image of the second marker M2 interfering with the image of the measurement site in the radiographic image. Providing the second marker M2 outside of the first region 120 can improve the alignment accuracy of the morphological image with respect to the biomagnetic measurement result without hindering the biomagnetic measurement.

FIG. 4C illustrates another example of the positional relationship between the arrangement of FIG. 4A. The first region 120 and the second region 150 may partially overlap one another. For example, when magnetic measurement of a target measurement site (such as an arm) placed on the bridge 14B is to be performed by using a portion of the magnetic sensor array 121, the second region 150 may be set to partially overlap the first region 120 so that the target measurement site and the second marker M2 are visible. Even in such a case, the first marker M1 is provided in the first region 120 that is detectable by the biomagnetic detector 12, and the second marker M2 (including markers M21 and M22) is provided so as to be outside of the first region 120 and inside of the second region 150.

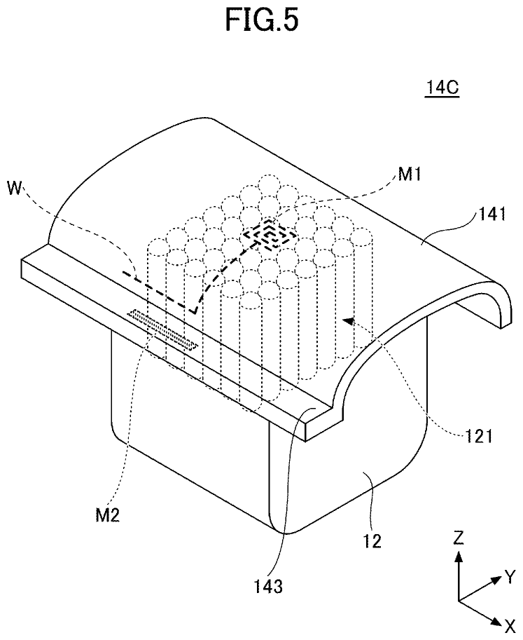

FIG. 5 illustrates yet another example of the first marker M1 and the second marker M2 provided in a bridge 14C. The bridge 14C includes a body 141 that faces the biomagnetic detector 12 and the flat portion 143 that extends from the body 141. The first marker M1 is provided at substantially the center of the body 141. The second marker M2 is embedded in the flat portion 143 of the bridge 14C. The second marker M2 is a linear non-magnetic metal pattern or a bar-shaped mark having a predetermined length. The length of the second marker M2 in the spatial coordinate system is known or obtainable.

The second marker M2 appears as a line segment in the radiographic image. The image data obtained from radiographic imaging can be enlarged or reduced to the scale of the biomagnetic measurement result based on a length L1 of the second marker M2 in the flat portion 143 and a length L2 of the line segment in the radiographic image. The relative positional relationship of the second marker M2 with respect to the first marker M1 is known. After the image data is enlarged or reduced, the relative position of the second marker M2 in the radiographic image can be aligned with respect to the position of the first marker M1 that was estimated from the biomagnetic measurement result. As a result, the morphological image can be accurately superimposed onto the biomagnetic measurement result.

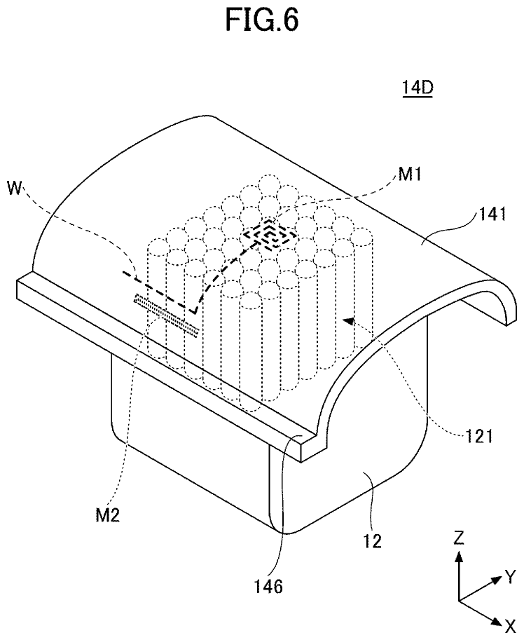

FIG. 6 illustrates yet another example of the first marker M1 and the second marker M2 provided in a bridge 14D. The bridge 14D includes the body 141 that faces the biomagnetic detector 12 and the fixing portion 146 that extends from the body 141. The body 141 may include a curved surface that conforms to the shape of the upper surface of the biomagnetic detector 12. The fixing portion 146 has a shape and a dimension that enable it to be fixed to the bed 13.

The first marker M1 and the second marker M2 are provided in the body 141. The first marker M1 is provided at substantially the center of the body 141 where the magnetic field is easily detected. The second marker M2 is embedded in an end portion of the body 141 such that the second marker M2 is outside of the magnetic detection region. Placing the first marker M1 in the first region 120 where the magnetic detection sensitivity is favorable allows the position of the first marker M1 in the measured magnetic field data to be estimated accurately. Providing the second marker M2 to be outside of the magnetic detection region (the first region) of the biomagnetic detector 12 and to be inside of the imaging region (the second region) allows the relative position of the second marker M2 with respect to the first marker M1 to be obtained without interfering with, for example, bones or joints that are captured in the radiographic image.

The second marker M2 can be used to enlarge or reduce the morphological image suitably, thus allowing the converted morphological image to be accurately superimposed onto the biomagnetic measurement result.

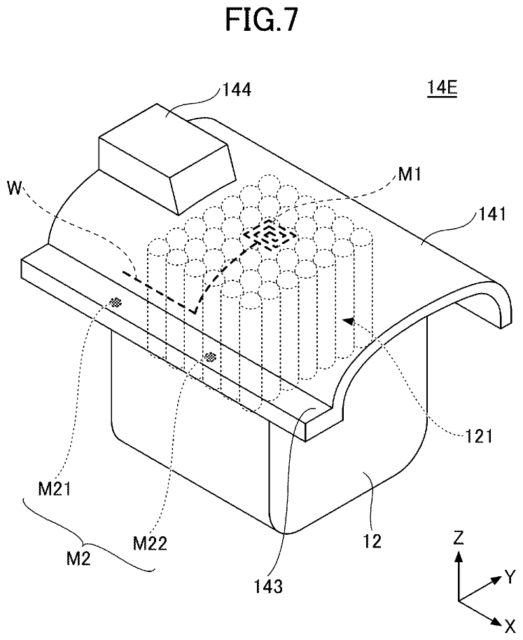

FIG. 7 illustrates the configuration of a bridge 14E according to a modification. The bridge 14E includes a protrusion 144 on the surface of the body 141. The protrusion 144 is provided in a position that does not interfere with the magnetic detection region of the magnetic sensor array 121. The protrusion 144 is used to fix the position of the measurement site of the subject S. For example, the subject S can lie supine with the side surface of the neck pressed against the protrusion 144, thus enabling the position of the measurement site to be kept constant with respect to the magnetic sensor array 121. Further, the posture of the measurement site of the subject S can be kept constant throughout the processes of biomagnetic measurement and radiographic imaging.

Although the bridge 14E is provided with the flat portion 143 and the two markers M21 and M22 are embedded in the flat portion 143 in FIG. 7, the bridge shape with the protrusion 144 can be combined with any of the marker arrangements illustrated in FIGS. 4A to 6.

<Biomagnetic Measuring System>

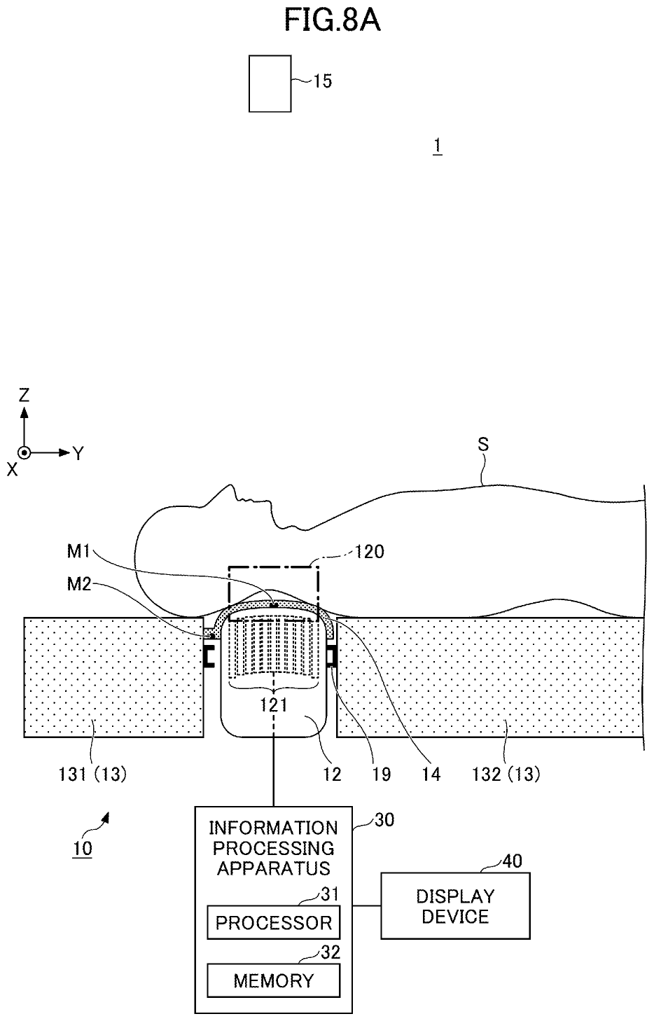

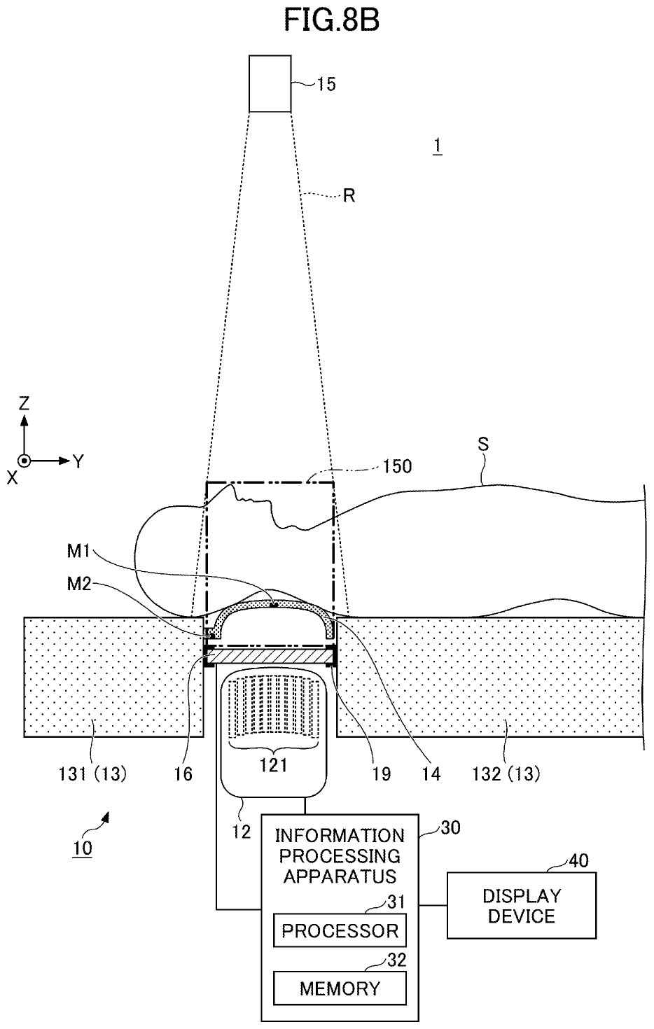

FIG. 8A is a schematic view of a biomagnetic measuring system 1 according to the embodiment during biomagnetic measurement. FIG. 8B is a schematic view of the biomagnetic measuring system 1 during radiographic imaging. The biomagnetic measuring system 1 includes the biomagnetic measuring apparatus 10, an information processing apparatus 30, and a display device 40. The display device 40 may be provided externally to the information processing apparatus 30 or integrated into the information processing apparatus 30. The information processing apparatus 30 includes a processor 31 and a memory 32, and is connected to the biomagnetic detector 12 and the radiation detector 16.

In FIG. 8A, the bed 13 is in the lowered position and the biomagnetic detector 12 is in close contact with the bridge 14 during biomagnetic measurement. The information processing apparatus 30 receives magnetic signals that are output from the individual magnetic sensors of the magnetic sensor array 121. The magnetic signals output from the biomagnetic detector 12 include the magnetic field information of the skin surface of the measurement site measured in the first region 120 and the magnetic field information from the first marker M1. The magnetic signals may be input directly from the biomagnetic detector 12 to the information processing apparatus 30 to store the input magnetic signals as magnetic field data in the memory 32. Alternatively, a data logger may be connected between the biomagnetic detector 12 and the information processing apparatus 30, and the magnetic signals accumulated in the data logger can be input as the magnetic field data in the information processing apparatus 30.

In FIG. 8B, the bed 13 is in the raised position and the radiation detector 16 has been set between the bridge 14 and the biomagnetic detector 12 during radiographic imaging. The radiation detector 16 is connected to the information processing apparatus 30. The imaging data obtained from the radiation detector 16 are input to the information processing apparatus 30 and are stored in the memory 32. The processor 31 of the information processing apparatus 30 obtains, based on the second marker M2 in the imaging data, a factor for enlarging or reducing the morphological image with respect to the biomagnetic measurement result. Subsequently, the processor 31 generates a converted morphological image by using the obtained factor to convert the morphological image into the scale of the biomagnetic measurement result.

The processor 31 also aligns, based on the positional relationship between the first marker M1 and second marker M2, the position of the second marker M2 in the morphological image with respect to the position of the first marker M1 that has been estimated from the magnetic field data. Subsequently, the processor 31 superimposes the morphological image onto the magnetic field data or a current distribution estimated from the magnetic field data. The position of the first marker M1 in the magnetic field data can be obtained by solving, based on the amplitude and phase of the magnetic field waveform from the first marker M1, an inverse problem by a known method such as an optimization algorithm. The image in which the morphological image is superimposed onto the biomagnetic measurement result or an analysis image thereof is output and displayed on the display device 40.

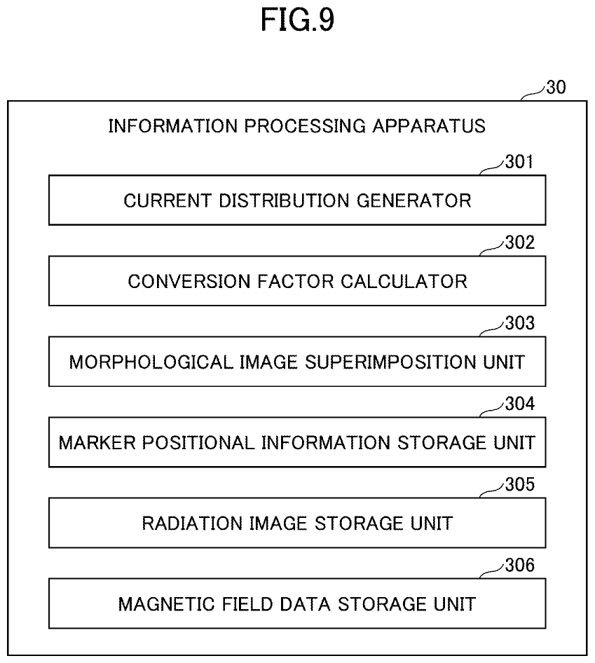

FIG. 9 illustrates a functional block diagram of the information processing apparatus 30. The information processing apparatus 30 includes a current distribution generator 301, a conversion factor calculator 302, a morphological image superimposition unit 303, a marker positional information storage unit 304, a radiographic image storage unit 305, and a magnetic field data storage unit 306. The respective functions of the current distribution generator 301, the conversion factor calculator 302, and the morphological image superimposition unit 303 are implemented by the processor 31. The respective functions of the marker positional information storage unit 304, the radiographic image storage unit 305, and the magnetic field data storage unit 306 are implemented by the memory 32.

The marker positional information storage unit 304 stores information related to the positional relationship of the first marker M1 and the second marker M2 that has been measured in advance or obtained from an external device. The radiographic image storage unit 305 stores the radiographic image obtained by the radiation detector 16. The radiographic image includes the radiographic image of the second marker M2. The magnetic field data storage unit 306 stores the magnetic field data obtained from the biomagnetic detector 12. As described above, the magnetic field data includes the magnetic field information of the surface of the measurement site of the subject S and the magnetic field information generated by the first marker M1. In a case where the amount by which the bed 13 is raised is to be obtained based on the change in the magnetic field of the first marker M1, the magnetic field data may include the magnetic field information of the first marker M1 at the raised position (imaging position) of the bed 13.

The conversion factor calculator 302 uses the second marker M2, which is included in the radiographic image stored in the radiographic image storage unit 305, and the positional information, which is stored in the marker positional information storage unit 304, to calculate a factor for enlarging or reducing the radiographic image. The morphological image superimposition unit 303 generates a morphological image by converting, based on the obtained enlargement factor or the reduction factor, the scale of the radiographic image into the scale of the coordinate system. Based on the relative positional information of the second marker M2 with respect to the first marker M1, the morphological image superimposition unit 303 aligns the position of the second marker M2 in the morphological image relative to the position of the first marker M1 that has been estimated from the magnetic field data. As a result, the morphological image is superimposed onto the magnetic field data that is the biomagnetic measurement result.

The current distribution generator 301 uses an estimation method such as spatial filtering to estimate a magnetic field source, that is, the current distribution within the living body from the magnetic field data within a boundary determined by the contour of the morphological image. The current distribution generator 301 subsequently generates a reconfigured current surface. The reconfigured current surface is output and displayed on the display device 40.

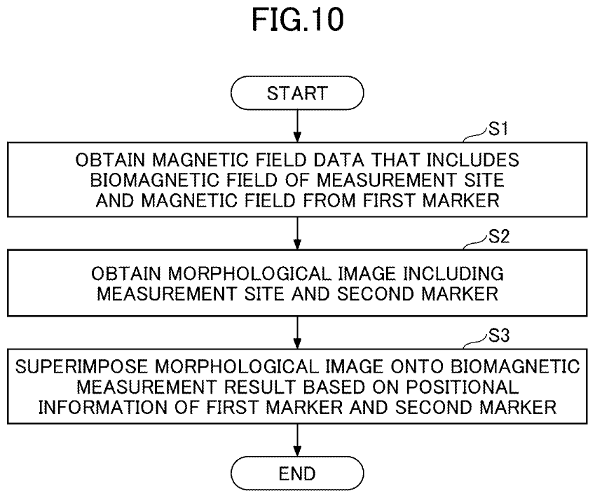

FIG. 10 is a flowchart of a biomagnetic measuring method according to the embodiment. The processes of this flowchart are executed by the information processing apparatus 30. The information processing apparatus 30 obtains, as a biomagnetic measurement result, the magnetic field data including the biomagnetic field of the measurement site of the subject S and the magnetic field from the first marker M1 (step S1). Further, the information processing apparatus 30 obtains a morphological image that includes the measurement site of the subject S and the second marker M2 (step S2). Steps S1 and S2 can be performed in any suitable order. The biomagnetic measurement result is obtained from the biomagnetic detector 12. The morphological image is obtained from the radiation detector 16.

The information processing apparatus 30 superimposes the morphological image onto the magnetic field data, that is, the biomagnetic measurement result based on the positional information of the first marker M1 and the second marker M2 stored in the memory 32 or obtained from an external device (step S3). The boundary of the magnetic field data measured in the measurement site is determined as a result. Subsequently, the magnetic field source, that is, the distribution of the current source in the living body may be analyzed within the boundary and may be output.

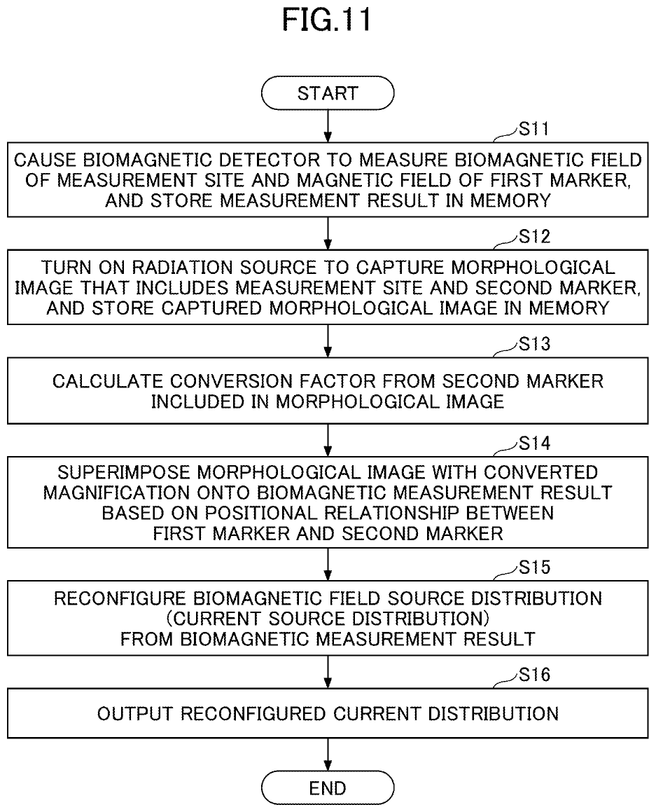

FIG. 11 illustrates a more detailed flowchart of the biomagnetic measuring method. The processes of this flowchart are executed by the biomagnetic measuring system 1. The biomagnetic detector 12 measures the biomagnetic field of the measurement site of the subject S and the magnetic field generated by the first marker M1, and the measurement result is stored in the memory 32 of the information processing apparatus 30 (step S11). The radiation detector 16 is set and the radiation source is turned on to capture a morphological image that includes the measurement site and the second marker M2, and the morphological image is stored in the memory 32 of the information processing apparatus 30 (step S12). Steps S11 and S12 can be performed in any suitable order. In a case where the process of step S11 is to be performed first, the bed 13 may be raised to set the radiation detector 16 on the mount 19, and radiographic imaging may be performed in the raised position of the bed 13. In a case where the process of step S12 is to be performed first, the radiation detector 16 is removed from the mount 19 after the radiographic imaging, the bed 13 is manipulated such that the bridge 14 is brought into close contact with the biomagnetic detector 12, and biomagnetic measurement is subsequently performed.

In the information processing apparatus 30, the conversion factor that is to be used to acquire correspondence with the biomagnetic measurement result is calculated from the second marker M2 included in the morphological image, and the morphological image is enlarged or reduced based on the obtained conversion factor (step S13). The morphological image is superimposed onto the biomagnetic measurement result by aligning, based on the positional relationship between the first marker M1 and the second marker M2, the position of the second marker M2 in the scaled morphological image relative to the position of the first marker M1 that has been estimated from the magnetic field data (step S14). As a result, the measurement site is associated with the magnetic field obtained from the skin surface.

The information processing apparatus 30 analyzes the magnetic field within the boundary of the measurement site, and reconfigures the magnetic field distribution of the skin surface into the magnetic field distribution (current source distribution) inside the living body (step S15). The reconfigured current distribution is output and displayed (step S16).

In the biomagnetic measuring system 1, the magnetically detectable first marker M1 and the second marker M2 whose image can be captured by radiographic imaging are provided in the bridge 14. Hence, the positional relationship between the first marker M1 and the second marker M2 does not change, thus allowing the positional relationship to be obtained by a single measurement and be repeatedly used as a constant piece of positional information. Placing the first marker M1 within the detection range of the biomagnetic detector 12 improves the accuracy in the positional estimation of the first marker M1. Placing the second marker M2 in the imaging range so that the second marker M2 is outside of the magnetic detection range allows radiographic imaging to be performed at a position where the second marker M2 does not interfere with the measurement site. As a result, an image of the second marker M2 can be captured together with the measurement site. Since the magnetic field data and the morphological image are aligned based on the relative positional relationship between the first marker M1 and the second marker M2 that is obtained in advance, the morphological image can be accurately superimposed onto the biomagnetic measurement result even in a case where it is difficult to discriminate the first marker M1 in the morphological image.

Biomagnetic measurement has been described based on specific configurational examples. However, the present disclosure is not limited to the examples described above. Although it is preferable for the shape of the bridge 14 to conform to the upper surface of the biomagnetic detector 12, the surface shape of the bridge 14 need not completely match. A triangular mark or a rectangular mark may be used as the second marker M2 instead of a sphere or a linear pattern. Since the first marker M1 need only be able to generate a magnetic field through application of a current, the first marker M1 is not limited a spiral coil and may be a single loop. The biomagnetic measurement according to the embodiment is not limited to the measurement of a magnetic field signal from the cervical spinal cord, and is also applicable to the biomagnetic measurement of the cauda equina nerve and other nervous systems.

The radiation source 5 is provided above the biomagnetic detector 12 and the radiation detector 16 is provided at a position that faces the radiation source 5 in the embodiment. However, the configuration is not limited to this. Instead of, or in addition to, a configuration in which imaging of the subject S is performed from above, radiographic imaging may also be performed laterally with respect to the subject S. In such a case, the radiation source may be provided on one side (for example, the right side) and the radiation detector may be provided on the opposite side (for example, the left side) with the subject S interposed therebetween.

What is claimed is:

1. A biomagnetic measuring apparatus comprising:
a biomagnetic detector;
a first marker configured to be detectable by the biomagnetic detector;
a radiation source;
a radiation detector provided to face the radiation source; and
a second marker configured such that an image of the second marker can be captured by the radiation source and the radiation detector,
wherein positional information about the first marker and the second marker is known or obtainable.

2. The biomagnetic measuring apparatus according to claim 1, wherein the first marker is provided in a first region that can be detected by the biomagnetic detector,
wherein the second marker is provided in a second region where an image can be captured, the second region being determined by a positional relationship between the radiation source and the radiation detector, and
the first region and the second region partially overlap one another.

3. The biomagnetic measuring apparatus according to claim 2, wherein within a two-dimensional plane parallel to a magnetic detection surface of the biomagnetic detector or an imaging surface of the radiation detector, an area of the first region is smaller than an area of the second region, and the second marker is positioned outside of the first region and inside of the second region.

4. The biomagnetic measuring apparatus according to claim 1, wherein the second marker is a plurality of second markers that are separated from each other by a predetermined distance, and
wherein respective coordinate positions of the plurality of second markers or a center-to-center distance between the plurality of second markers is known.

5. The biomagnetic measuring apparatus according to claim 1, wherein the second marker is a linear or bar-shaped marker having a predetermined length, and the length of the second marker is known.

6. The biomagnetic measuring apparatus according to claim 1, further comprising:
a bed with a space in which at least a portion of the biomagnetic detector is contained; and
a bridge facing the biomagnetic detector across the space,
wherein the first marker and the second marker are provided on the bridge.

7. The biomagnetic measuring apparatus according to claim 6, wherein the bridge includes a body facing the biomagnetic detector and a flat portion extending from the body, and
wherein the first marker is provided in the body, and
wherein the second marker is provided in the flat portion.

8. The biomagnetic measuring apparatus according to claim 6, wherein the bridge includes a body facing the biomagnetic detector,
wherein the first marker is provided in a center of the body or near the center of the body, and
wherein the second marker is provided at an end portion of the body.

9. The biomagnetic measuring apparatus according to claim 1, wherein the radiation source is an X-ray radiation source, and
wherein the second marker has a lower X-ray transmittance than the first marker.

10. The biomagnetic measuring apparatus according to claim 1, wherein the first marker is a flexible printed circuit with a coiled pattern.

11. A biomagnetic measuring system comprising:
the biomagnetic measuring apparatus of claim 1;
an information processing apparatus configured to be connected to the biomagnetic measuring apparatus; and
a display device provided in the information processing apparatus or configured to be connected to the information processing apparatus,
wherein the information processing apparatus is configured to combine a biomagnetic measurement result obtained from the biomagnetic detector and an imaging result obtained by the radiation source and the radiation detector, and to display the combined result on the display device.

12. A biomagnetic measuring method comprising:
obtaining magnetic field data including a biomagnetic field of a measurement site and a magnetic field from a first marker provided in a magnetic measurement region;
obtaining a morphological image including the measurement site and a second marker provided in an imaging region; and
superimposing, based on positional information of the first marker and the second marker, the magnetic field data onto the morphological image or the morphological image onto the magnetic field data.
